(19) United States
(12) Patent Application Publication
(10) Pub. No.: US 2006/0110805 A1
(43) Pub. Date: May 25, 2006

(54) Microbial production of xylitol via hexose phosphate and pentose phosphate intermediate

(75) Inventors: Ian Fotheringham, Schaumburg, IL (US); Paul Taylor, Arlington Heights, IL (US); David Demirjian, Hinsdale, IL (US); Nick Oswald, Edinburgh (GB)

Correspondence Address: MCDONNELL BOEHNEN HULBERT & BERGHOFF LLP, 300 S. WACKER DRIVE, 32ND FLOOR, CHICAGO, IL, 60606, US

(73) Assignees: Biotechnology Research and Development Corporation; Agricultural Research Service, United States Department of Agriculture

(21) Appl. No.: 11/133,025

(22) Filed: May 19, 2005

Related U.S. Application Data
(60) Provisional application No. 60/572,588, filed on May 19, 2004.
(60) Provisional application No. 60/620,173, filed on Oct. 18, 2004.
(60) Provisional application No. 60/572,438, filed on May 19, 2004.

(51) Int. Cl. C12P 19/02; C12P 7/18; C12N 1/21; C12N 1/18

(52) U.S. Cl. 435/105; 435/252.3; 435/254.23; 435/252.31; 435/252.33; 435/252.34; 435/158

(57) ABSTRACT

The invention provides methods and compositions for production of xylitol, xylose, or combinations thereof from simple sugars from biosynthetic pathways that utilize D-fructose-6-phosphate and D-xylulose-5-phosphate as pathway intermediates.

### PRIORITY

This application claims priority to U.S. application Ser. No. ______, filed May 19, 2005. This application also claims the benefit of U.S. Provision Application Ser. Nos. 60/572,588, filed May 19, 2004; 60/620,173, filed Oct. 18, 2004; and 60/572,438, filed May 19, 2004, all of which are incorporated by reference herein in their entirety.

### FIELD OF THE INVENTION

The invention is in the field of constructing an effective biosynthetic route to xylitol that utilizes D-fructose-6-phosphate and D-xylulose-5-phosphate as pathway intermediates and simple sugars such as a carbon source.

### BACKGROUND TO THE INVENTION

Xylitol is currently produced by chemical hydrogenation of xylose purified from xylan hydrolysates. The use of microorganisms to produce xylitol and other polyols from inexpensive starting materials such as corn and other agricultural byproduct and waste streams has long been thought to be able to significantly reduce production costs for these polyols as compared to chemical hydrogenation. Such a process would reduce the need for purified xylose, produce purer, easier to separate product, and be adaptable to a wide variety of raw materials from different geographic locations.

Despite a significant amount of earlier work, development of a commercially feasible microbial production process has remained elusive for a number of reasons. To date, even with the advent of genetically engineered yeast strains, the volumetric productivity of the strains developed do not reach the levels necessary for a commercial process. This invention relates to the development of whole-cell microbial processes using enzyme systems capable of converting D-Glucose to xylitol

Xylitol is currently produced from plant materials—specifically hemicellulose hydrolysates. Different plant sources contain different percentages of cellulose, hemicellulose, and lignin making most of them unsuitable for xylitol production.

Because of purity issues, only the hydrolysate from birch trees is used for xylitol production. Birch tree hydrolysate is obtained as a byproduct of the paper and pulping industry, where lignins and cellulosic components have been removed. Hydrolysis of other xylan-rich materials, such as trees, straws, corncobs, oat hulls under alkaline conditions also yields hemicellulose hydrolyzate, however these hydrolyzates contain many competing substrates. One of these substrates, L-arabinose is a particular problem to xylitol production because it can be converted to L-arabinitol, which is practically impossible to separate from xylitol in a cost effective way.

D-xylose in the hydrolysate is converted to xylitol by catalytic reduction. This method utilizes highly specialized and expensive equipment for the high pressure (up to 50 atm) and temperature (80-140° C.) requirements as well as the use of Raney-Nickel catalyst that can introduce nickel into the final product. There have been several processes of this type described previously, for example U.S. Pat. Nos. 3,784,408, 4,066,711, 4,075,406, 4,008,285, and 3,586,537. In addition, the xylose used for the chemical reduction must be substantially purified from lignin and other cellulosic components of the hemicellulose hydrolysate to avoid production of extensive by-products during the reaction.

The availability of the purified birch tree hydrolysate starting material severely limits the xylitol industry today. If an efficient reduction process could be developed that could convert an alternative starting material such as D-glucose to xylitol, then a highly cost competitive process could be developed from an abundant and inexpensive starting material that would allow significant expansion of the xylitol market.

Many of the prior art methods of producing xylitol use purified D-xylose as a starting material and will also generally convert L-arabinose to L-arabitol (and other sugars to their respective reduced sugar polyol). While there has been a significant amount of work on the development of an organism to convert D-xylose to xylitol, none of the prior art approaches have been commercially effective. There are several reasons for this. First, D-xylose utilization is often naturally inhibited by the presence of glucose that is used as a preferred carbon source for many organisms. Second, none of the enzymes involved have been optimized to the point of being cost effective. Finally, D-xylose in its pure form is expensive. Prior art methods do not address the need for alternative starting materials. Instead they require relatively pure D-xylose. Agricultural waste streams are considered to be the most cost-effective source of xylose. These waste streams are generally mixed with a variety of other hemicellulosic sugars (L-arabinose, galactose, mannose, and glucose), which all affect xylitol production by the microbes in question. See, Walthers et al. (2001). “Model compound studies: influence of aeration and hemicellulosic sugars on xylitol production by Candida tropicalis.” Appl Biochem Biotechnol 91-93:423-35. However, if an organism can be engineered to utilize an abundant and inexpensive starting material such as D-glucose it would make the process much more cost effective.

A variety of approaches have been reported in the literature for the biological production of xylitol. While some basic research has been performed, development of an effective bioprocess for the production of xylitol has been elusive. Many of the systems described below suffer from problems such as poor strain performance, low volumetric productivity, and too broad of a substrate range. Of these, yeasts, primarily Candida, have been shown to be the best producers of xylitol from pure D-xylose. See, Hahn-Hagerdal, et al., Biochemistry and physiology of xylose fermentation by yeasts. Enzyme Microb. Technol., 1994. 16:933-943; Jeffries & Kurtzman, Strain selection, taxonomy, and genetics of xylose-fermenting yeasts. Enzyme Microb. Technol., 1994. 16:922-932; Kern, et al., Induction of aldose reductase and xylitol dehydrogenase activities in Candida tenuis CBS 4435. FEMS Microbiol Lett, 1997. 149(1):31-7; Saha & Bothast, Production of xylitol by Candida peltata. J Ind Microbiol Biotechnol, 1999. 22(6):633-636; Saha & Bothast, Microbial production of xylitol, in Fuels and Chemicals from Biomass, Saha, Editor. 1997, American Chemical Society. p. 307-319. These include Candida strains C. guilliermondii, C. tropicalis, C. peltata, C. milleri, C. shehatae, C. boidinii, and C. parapsilosis. C. guillermondii is one of the most studied organisms and has been shown to have a yield of up-to 75% (g/g) xylitol from a 300 g/l fermentation mixture of xylose. See, Saha & Bothast, Production of xylitol by Candida peltata. J Ind Microbiol Biotechnol, 1999. 22(6):633-636. C. tropicalis has also been shown to be a relatively high producer with a cell recycling system producing an 82% yield with a volumetric productivity of 5 g L⁻¹ h⁻¹ and a substrate concentration of 750 g/l. All of these studies however, were carried out using purified D-xylose as substrate.

Bolak Co., Ltd, of Korea describes a two-substrate fermentation with C. tropicalis ATCC 13803 using glucose for cell growth and xylose for xylitol production. The optimized fed-batch fermentation resulted in 187 g L⁻¹ xylitol concentration, 75% g/g xylitol/xylose yield and 3.9 g xylitol L⁻¹ H⁻¹ volumetric productivity. See, Kim et al., Optimization of fed-batch fermentation for xylitol production by Candida tropicalis. J Ind Microbiol Biotechnol, 2002. 29(1):16-9. The range of xylose concentrations in the medium ranged from 100 to 200 g L⁻¹ total xylose plus xylitol concentration for maximum xylitol production rate and xylitol yield. Increasing the concentrations of xylose and xylitol beyond this decreased the rate and yield of xylitol production and the specific cell growth rate, and the authors speculate that this was probably due to the increase in osmotic stress. Bolak disclosed this approach to xylitol production. See e.g., U.S. Pat. No. 5,998,181; U.S. Pat. No. 5,686,277. They describe a method of production using a novel strain of Candida tropicalis KCCM 10122 with a volumetric productivity in 3 to 5 L reactions ranging from 3.0 to 7.0 g xylitol L⁻¹ H⁻¹, depending on reaction conditions. They also describe a strain, Candida parapsilosis DCCM-10088, which can transform xylose to xylitol with a maximum volumetric productivity of 4.7 g xylitol L⁻¹ H⁻¹, again in bench scale fermentation ranging from 3 to 5 liters in size. While C. tropicalis has had moderate success in achieving relatively large levels of xylitol production than the other strains, it suffers from the fact that it is an opportunistic pathogen, and therefore is not suitable for food production and the enzyme also makes L-arabitol from L-arabinose.

One promising approach that has only been moderately explored is the creation of recombinant strains capable of producing xylitol. Xyrofin has disclosed a method involving the cloning of a xylose reductase gene from certain yeasts and transferring the gene into a Saccharomyces cerevisiae. See, U.S. Pat. No. 5,866,382. The resulting recombinant yeast is capable of reducing xylose to xylitol both in vivo and in vitro. An isolated enzyme system combining xylitol reductase with formate dehydrogenase to recycle the NADH cofactor during the reaction has been described. In this instance, the enzymatic synthesis of xylitol from xylose was carried out in a fed-batch bioreactor to produce 2.8 g/l xylitol over a 20 hour period yielding a volumetric productivity of about 0.4 g l⁻¹ H⁻¹. See, Neuhauser et al., A pH-controlled fed-batch process can overcome inhibition by formate in NADH-dependent enzymatic reductions using formate dehydrogenase-catalyzed coenzyme regeneration. Biotechnol Bioeng, 1998. 60(3):277-82. The use of this on a large scale using crude substrate has yet to be demonstrated and poses several technical hurdles.

Several methods for producing xylitol from xylose-rich lignocellulosic hydrolyzates through fermentative processes have been described. Xyrofin discloses a method for the production of substantially pure xylitol from an aqueous xylose solution. See, U.S. Pat. No. 5,081,026; U.S. Pat. No. 5,998,607. This solution may also contain hexoses such as glucose. The process uses a yeast strain to convert free xylose to xylitol while the free hexoses are converted to ethanol. The yeast cells are removed from the fermentation by filtration, centrifugation or other suitable methods, and ethanol is removed by evaporation or distillation. Chromatographic separation is used to for final purification. The process is not commercially viable because it requires low arabinose wood hydrolyzate to prevent L-arabitol formation and the total yield was (95 g l⁻¹) and volumetric productivity is low (1.5 g l⁻¹ H⁻¹). Xyrofin also discloses a method for xylitol synthesis using a recombinant yeast (Zygosaccharomyces rouxii) to convert D-arabitol to xylitol. See, U.S. Pat. No. 5,631,150. The recombinant yeast contained genes encoding D-arbinitol dehydrogenase (E.C. 1.1.1.11) and xylitol dehydrogenase (E.C. 1.1.1.9), making them capable of producing xylitol when grown on carbon sources other than D-xylulose or D-xylose. The total yield (15 g l⁻¹) and volumetric productivity (0.175 g l⁻¹ H⁻¹) coupled with the use of D-arabitol as starting material make this route highly unlikely to succeed. Additionally, a 2-step fermentation of glucose to D-arabitol followed by fermentation of D-arabitol to xylitol has also been described. See, U.S. Pat. No. 5,631,150; U.S. Pat. No. 6,303,353; U.S. Pat. No. 6,340,582. However, a two-step fermentation is not economically feasible.

Another method of making xylitol using yeasts with modified xylitol metabolism has been described. See, U.S. Pat. No. 6,271,007. The yeast is capable of reducing xylose and using xylose as the sole carbon source. The yeast have been genetically modified to be incapable or deficient in their expression of xylitol dehydrogenase and/or xylulose kinase activity, resulting in an accumulation of xylitol in the medium. A major problem with this method is that a major proportion of the D-xylose is consumed for growth rather than being converted to the desired product, xylitol.

Ajinomoto has several patents/patent applications concerning the biological production of xylitol. In U.S. Pat. No. 6,340,582, they claim a method for producing xylitol with a microorganism containing D-arbinitol dehydrogenase activity and D-xylulose dehydrogenase activity. This allows the organisms to convert D-arbinitol to D-xylulose and the D-xylulose to xylitol, with an added carbon source for growth. Sugiyama further develops this method in U.S. Pat. No. 6,303,353 with a list of specific species and genera that are capable of performing this transforming, including Gluconobacter and Acetobacter species. This work is furthered by the disclosure of the purified and isolated genes for two kinds of xylitol dehydrogenase from Gluconobacter oxydans and the DNA and amino acid sequences, for use in producing xylitol from D-xylulose. See, U.S. Pat. Publ. 2001/0034049; U.S. Pat. No. 6,242,228. In US Appl. Publ. No. 2003/0148482 they further claim a microorganism engineered to contain a xylitol dehydrogenase, that has an ability to supply reducing power with D-xylulose to produce xylitol, particularly in a microorganism that has an ability to convert D-arbinitol into D-xylulose.

Ajinomoto has also described methods of producing xylitol from glucose. Takeuchi et al. in U.S. Pat. No. 6,221,634 describes a method for producing either xylitol or D-xylulose from Gluconobacter, Acetobacter or Frateuria species from glucose.

However, yields of xylitol were less than 1%. Mihara et al. further claim specific osmotic stress resistant Gluconobacter and Acetobacter strains for the production of xylitol and xylulose from the fermentation of glucose. See, U.S. Pat. No. 6,335,177. They report a 3% yield from a 20% glucose fermentation broth. In U.S. Pat. Appl. No. 2002/0061561, Mihara et al. claim further discovered strains, also with yields of only a few percent. See, U.S. Pat. No. 6,335,177.

Cerestar has disclosed a process of producing xylitol from a hexose such as glucose in two steps. See, U.S. Pat. No. 6,458,570. The first step is the fermentative conversion of a hexose to a pentitol, for example, glucose to arabitol, and the second step is the catalytic chemical isomerisation of the pentitol to xylitol.

Bley et al. disclose a method for the biotechnological production of xylitol using microorganisms that can metabolize xylose to xylitol. See, WO03/097848. The method comprises the following steps: a) microorganisms are modified such that oxidation of NADH by enzymes other than the xylose reductase is reduced or excluded; b) the microorganisms are cultivated in a substrate containing xylose and 10-40 grams per liter of sulphite salt (e.g. calcium hydrogen sulphite, natrium sulphite, potassium sulphite); c) the microorganisms are cultivated in an aerobic growth phase and an oxygen-limited xylitol production phase; and d) the xylitol is enriched and recovered from the substrate.

Londesborough et al. have disclosed a genetically modified fungus containing L-arabitol 4-dehydrogenase and L-xylulose xylulose reductase. See, U.S. Pat. Appl. Publ. No. 2003/0186402. This application is aimed at producing useful products from biomass containing L-arabinose, which is a major constituent of plant material but does not disclose the use of D-xylose/L-arabinose mixtures for the synthesis of xylitol in procaryotes. Verho et al. also describe and alternative L-xylulose reductase from Ambrosiozyma monospora that utilizes NADH as co-factor. See, Verho et al., New Enzyme for an in vivo and in vitro Utilization of Carbohydrates. 2004, Valtion Teknillinen Tutki-muskeskus. p. 15.

Researchers at Danisco have developed several xylitol bioprocesses. Heikkila et al. describes a process wherein purified L-xylose is utilized as intermediate. See, U.S. Pat. Appl. Publ. No. 2003/0097029. The application also covers methods of production of L-xylose. This process is not feasible because L-xylose is a rare sugar and is considerably more valuable than the final product. A method for simultaneously producing xylitol as a co-product during fermentative ethanol production, utilizing hydrolyzed lignocellulose-containing material is disclosed in U.S. Pat. Appl. Publ. No. 2003/0235881. This process consists of fermenting the free hexoses to ethanol while the xylose is converted to xylitol with a single yeast strain. The yields, however, of both ethanol and xylitol were relatively poor and require pure D-xylose as a substrate. Danisco has also developed a multiple processes for the preparation of xylitol, all of them utilizing ribulose. See, U.S. Pat. Appl. Publ. No. 2003/0125588. These processes include different conversion reactions, such as reduction, epimerization and/or isomerisation. Xylitol is also produced in the fermentation of glucose in one embodiment. The process can also use ribulose and xylulose as starting material, followed by reduction, epimerization and isomerisation to xylitol. Again the starting substrates D-xylulose and ribulose are more valuable than the final product.

Ojamo et al. shows a method for the production of xylitol involving a pair of microorganisms one having xylanolytic activity, and another capable of converting a pentose sugar to xylitol, or a single microorganism capable of both reactions. See, U.S. Pat. Appl. Publ. No. 2004/0014185. In one embodiment of the invention, two microorganisms are used for the production of xylitol, one microorganism possessing xylanolytic activity and the other possessing the enzymatic activity needed for conversion of a pentose sugar, such as D-xylose and L-arabinose, preferably D-xylose, to xylitol. This method requires a complicated two-organism system and produces mixtures of xylitol and L-arabitol, which need extra purification and recycle steps to improve the xylitol yield. It does not teach simple, single organism methods that can use D-xylose/L-arabinose mixtures to synthesize pure xylitol. Finally, Miasnikov et al. have developed multiple methods for the production of xylitol, five-carbon aldo- and keto-sugars and sugar alcohols by fermentation in recombinant hosts. See, U.S. Pat. Appl. Publ. No. 2003/0068791. These recombinant hosts have been engineered to redirect pentose phosphate pathway intermediates via ribulose-5-P, xylulose-5-P and xylitol-5-P into the production of xylitol, D-arbinitol, D-arabinose, D-xylose, ribitol, D-ribose, D-ribulose, D-xylose, and/or D-xylulose. Methods of manufacturing are disclosed that use such hosts, but the productivity is low.

While clearly there has been a significant amount of work on the development of an organism to convert xylose to xylitol, none of these have resulted in an effective production organism or a commercialized process. The yeast methods described above all require relatively pure xylose as a starting material, since the organisms described will also convert L-arabinose to L-arabitol (and other sugars to their respective reduced sugar pentitol). This results in difficult-to-remove by-products which can only be separated by costly separation methods. Purified xylose is also prohibitively expensive for use in a bioprocess and cannot compete with the current chemical hydrogenation. Several of the processes above consist of more than one fermentation step, which is again, cost-prohibitive. The reported production rate of some of the strains is low, as in the Ajinomoto patents. Above all, none of the enzymes or strains involved has been engineered to be cost effective. If the turnover rate of one or more enzyme can be improved, then the production level would increase. Further, none of the approaches have addressed the problems associated with the use of agricultural hydrolyzates to produce xylitol. While agricultural waste streams are considered to be a cost-effective source of D-xylose, these waste streams are generally mixed with a variety of other hemicellulosic sugars (arabinose, galactose, mannose, and glucose), which all affect xylitol production by the microbes in question. See, Walthers et al., Model compound studies: influence of aeration and hemicellulosic sugars on xylitol production by Candida tropicalis. Appl Biochem Biotechnol, 2001. 91-93:423-35.

Thus a fermentation process that converts D-glucose to xylitol has the potential to replace the current methods of production and relieve the current limitations imposed by the limited supply of D-xylose.

Another reason that a highly efficient and economic process has not been able to be achieved has been the lack of powerful genetic tools such as screens and selections that can be used in a protein engineering and metabolic engineering program to rapidly identify high level producers of both xylitol and intermediates in the production of xylitol.

### BRIEF DESCRIPTION OF THE DRAWINGS

FIG. 1 shows a pathway for xylitol synthesis from D-glucose.

FIG. 2 shows the genotype and phenotype of a high throughput cross-feeding strain.

FIG. 3a shows the pINGE2 cloning vector.

FIG. 3b shows pING205 containing an xylAB operon and tkt gene.

FIG. 4a shows the pING211 expression vector.

FIG. 4b shows the pING210 expression vector.

### SUMMARY OF THE INVENTION

One embodiment of the invention provides a recombinant microorganism comprising a recombinant biochemical pathway to produce xylose or xylitol from fermentation of D-glucose.

Another embodiment of the invention provides a recombinant microorganism comprising transketolase, xylulokinase, xylose isomerase, and xylose reductase activities, wherein one or more of the transketolase, xylulokinase, xylose isomerase, and xylose reductase activities are elevated as compared to a wild-type microorganism, and wherein the recombinant microorganism can produce an end-product of xylose, xylitol, or a combination thereof from a substrate comprising D-glucose. The microorganism can further comprise xylitol dehydrogenase activity, which can be elevated as compared to a wild-type microorganism. The xylose isomerase activity can be reduced or eliminated as compared to a wild-type microorganism. The microorganism can have phosphofructokinase activity eliminated by disruption of the relevant genes or genes. The microorganism can be a bacterium, yeast or fungus. The microorganism can be Escherichia, Bacillus, Pseudomonas, Rhodococcus, or Actinomyces.

The recombinant microorganism can comprise one or more recombinant nucleic acid sequences encoding transketolase, xylulokinase, xylose isomerase, and xylose reductase or one or more recombinant nucleic acid sequences encoding transketolase, xylulokinase, xylose isomerase, xylose reductase and xylitol dehydrogenase.

The recombinant nucleic acid sequence encoding xylose reductase can be a Pichia stipitis nucleic acid sequence. The nucleic acid sequence encoding xylose reductase comprises a nucleic acid sequence encoding XYL1 from Candida tenuis. The nucleic acid sequence encoding xylose reductase can comprise a yafB or yajO nucleic acid sequence from E. coli. The nucleic acid sequence encoding xylitol dehydrogenase can be a Gluconobacter or Tricoderma reesi nucleic acid sequence. The nucleic acid sequence encoding transketolase can be a Escherichia coli tktA nucleic acid sequence. The nucleic acid sequence encoding xylose isomerase can be a Escherichia coli xylA nucleic acid sequence. The nucleic acid sequence encoding xylulokinase can be a Escherichia coli xylB or Saccharomyces cerevisiae XKS1 nucleic acid sequence. The nucleic acid sequence of another D-xylulose 5-phosphate dephosphorylating enzyme such as the nucleic acid sequence of dihydroxyacetone synthase from Mycobacterium sp., or Pichia angusta or alkaline phosphatase from Escherichia coli may be used along with or in place of xylulokinase. The nucleic acid sequence encoding xylose isomerase and xylulokinase can be an Escherichia coli xylAB operon. The recombinant microorganism can be non-pathogenic.

The recombinant microorganism can produce D-fructose-6-phosphate, D-xylulose-5-phosphate, D-xylulose, D-xylose, or combinations thereof as intermediates to the xylitol or xylose end-product.

Another embodiment of the invention provides a method for producing xylitol, xylose or a combination thereof end-product comprising fermenting a substrate comprising D-glucose with the recombinant microorganism comprising transketolase, xylulokinase (or other D-xylulose 5′-phosphate dephosphorylating activity), xylose isomerase, and xylose reductase activities, wherein one or more of the transketolase, xylulokinase, xylose isomerase, and xylose reductase activities are elevated as compared to a wild-type microorganism.

Still another embodiment of the invention provides a method for producing xylitol, xylose or combination thereof end-product comprising fermenting D-glucose with a recombinant microorganism comprising transketolase, xylulokinase, xylose isomerase, and xylose reductase activities, wherein one or more of the transketolase, xylulokinase, xylose isomerase, and xylose reductase activities are elevated as compared to a wild-type microorganism.

Yet another embodiment of the invention provides a recombinant indicator microorganism that expresses substantially no phosphotransferase enzyme I, expresses substantially no xylose isomerase, and has xylitol dehydrogenase activity. The indicator microorganism can be lac+. The recombinant indicator microorganism can comprise a recombinant nucleic acid sequence encoding xylitol dehydrogenase. The nucleic acid sequence encoding xylitol dehydrogenase can be a Gluconobacter or Tricoderma reesi nucleic acid sequence. The microorganism can be a bacteria, such as E. coli, yeast or fungi. The microorganism can be a phosphotransferase enzyme I deletion mutant or a xylose isomerase deletion mutant or both a phosphotransferase enzyme I deletion mutant and a xylose isomerase deletion mutant.

Even another embodiment of the invention provides a method of detecting production of xylose or xylitol from a sole carbon source, such as D-glucose or D-xylulose by a microorganism. The method comprises i) embedding a recombinant indicator microorganism that expresses substantially no phosphotransferase enzyme I, expresses substantially no xylose isomerase, and has xylitol dehydrogenase activity in a solid medium comprising D-glucose as a sole carbon source, ii) plating microorganisms to be tested on the solid media and incubating the solid media under conditions suitable for growth of the indicator microorganism and the microorganisms to be tested, wherein one or more plated colonies producing xylose or xylitol are visualized by growth of the indicator strain in an area surrounding the colony.

Even another embodiment of the invention provides a method of detecting the production of xylose from a sole carbon source such as D-glucose or D-xylulose by a microorganism. The method comprises i) embedding a recombinant indicator microorganism that expresses substantially no phosphotransferase enzyme I, expresses substantially no xylose isomerase, and has xylitol dehydrogenase activity in a solid medium comprising D-glucose as a sole carbon source, ii) plating microorganisms to be tested on the solid media and incubating the solid media under conditions suitable for growth of the indicator microorganism and the microorganisms to be tested, wherein one or more plated colonies producing xylose are visualized by growth of the indicator strain in an area surrounding the colony.

Another embodiment of the invention provides a method of detecting production of xylitol from a sole carbon source such as D-glucose or D-xylulose by a microorganism. The method comprises i) embedding a recombinant indicator microorganism that expresses substantially no phosphotransferase enzyme I, expresses substantially no xylose isomerase, and has xylitol dehydrogenase activity in a solid medium comprising D-glucose as a sole carbon source, ii) plating microorganisms to be tested on the solid media and incubating the solid media under conditions suitable for growth of the indicator microorganism and the microorganisms to be tested, wherein one or more plated colonies producing xylitol are visualized by growth of the indicator strain in an area surrounding the colony.

The microorganism to be tested can be subjected to random mutation using biological, chemical or physical means prior to the plating. The indicator microorganism can be lac⁺ and the microorganism to be tested for production of xylitol can be lac⁻ and the solid media can comprise X-gal. Areas of growth of the indicator microorganism are blue.

The beta-galactosidase enzyme of the indicator microorganism can be more tightly regulated than a wild-type beta-galactosidase enzyme by elevation of the intracellular level of a lactose repressor protein. The beta-galactosidase enzyme of the indicator microorganism can possess a shorter half-life than wild type beta-galactosidase due to alterations to its peptide sequence that decreases its stability under physiological conditions.

Still another embodiment of the invention provides a recombinant E. coli strain that produces substantially no phosphotransferase enzyme I and produces substantially no xylose isomerase. The E. coli strain can be a phosphotransferase enzyme I deletion mutant or a xylose isomerase deletion mutant or both a phosphotransferase enzyme I deletion mutant and a xylose isomerase deletion mutant.

Even another embodiment of the invention provides a method for screening for xylitol reductase activity. The method comprises transforming a recombinant E. coli strain that produces substantially no phosphotransferase enzyme I and produces substantially no xylose isomerase with a nucleic acid molecule encoding a putative xylose reductase to produce a transformant, and adding the transformant to a media comprising xylose as the sole carbon source. If the transformant comprises an expressed nucleic acid encoding a xylose reductase, then the transformant will grow in the media.

### DETAILED DESCRIPTION OF THE INVENTION

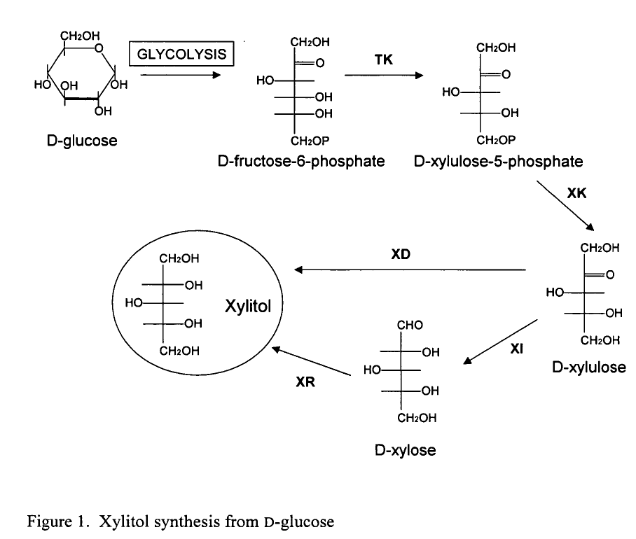

The instant invention provides methods and compositions for the synthesis of xylitol from sources including but not limited to, D-glucose, using a process or pathway shown in FIG. 1. In one embodiment of the invention, D-glucose is converted to D-fructose-6-phosphate via glycolysis. D-fructose-6-phosphate is converted to D-xylulose-5-phosphate by transketolase. D-xylulose-5-phosphate is converted to D-xylulose by xylulokinase or by other D-xylulose 5-phosphate dephosphorylating enzymes such as Pichia angusta or Mycobacterium sp. dihydroxyacetone synthase or a phosphatase such as the Escherichia coli alkaline phosphatase. D-xylulose can be converted to D-xylose by xylose isomerase. D-Xylose can be converted to xylitol by xylose reductase. Alternatively, D-xylulose can be converted to xylitol by xylitol dehydrogenase (XDH).

Alternatively, D-xylulose can be converted to both D-xylose and xylitol by xylose isomerase and xylitol dehydrogenase, respectively. The D-xylose can be converted to xylitol by xylose reductase.

In another embodiment of the invention, the starting material can be fructose or high fructose corn syrup. The fructose or high fructose corn syrup can be converted to fructose to glucose by a glucose isomerase. The glucose isomerase can be directly added to starting materials or intermediates or can be produced by a microorganism or a recombinant microorganism. A microorganism of the invention can produce glucose isomerase naturally or it can be a recombinant microorganism that expresses a recombinant glucose isomerase coding sequence. Glucose isomerase is well characterized and is widely distributed in prokaryotes. See, Bhosale et al., Microbiol. Rev. 1996. 60:280.

In one embodiment of the invention a recombinant microorganism comprises a recombinant biochemical pathway to produce xylose, xylitol, or xylulose from fermentation of dextrose. The recombinant microorganism comprises transketolase (TK), xylulokinase (XK), xylose isomerase (XI) and aldose (xylose) reductase (XR) activities.

In another embodiment of the invention a recombinant microorganism comprises a recombinant biochemical pathway to produce D-xylulose or xylitol from fermentation of D-glucose. The recombinant microorganism comprises transketolase (TK), xylulokinase (XK), and xylitol dehydrogenase (XDH) activities. One or more of the enzymes can be encoded by a recombinant nucleic acid. Additionally, one or more of the wild-type or recombinant nucleic acids encoding the enzymes can be engineered so that the enzymes are expressed at an elevated level as compared to any wild-type expression of the enzymes

A recombinant, isolated microorganism of the invention produces an end-product of xylitol or xylose or a combination thereof. One embodiment of the invention provides a recombinant microorganism comprising transketolase, xylulokinase, xylose isomerase, and xylose reductase activities and optionally xylitol dehydrogenase activity, wherein one or more of the transketolase, xylulokinase, xylose isomerase, xylose reductase and xylitol dehydrogenase activities are elevated as compared to a wild-type microorganism. The recombinant microorganism can produce an end-product of xylose or xylitol or a combination thereof from a substrate comprising, for example, D-glucose. An end product is a desired product that can accumulate in the growth medium of the producing culture or during a process with a minimal level of catabolism and that can be subsequently recovered.

In one embodiment of the invention a recombinant microorganism produces D-fructose-6-phosphate, D-xylulose-5-phosphate, D-xylulose, D-xylose, or combinations thereof as intermediates to the xylitol end-product. An intermediate product can be defined as a product generated from a starting substrate that requires further conversion into an end-product or that can be collected, processed, or removed separately from an end-product. The intermediate products can be collected before their ultimate conversion to xylitol if desired.

A recombinant microorganism of claim can be a bacterium, yeast or fungus. In one embodiment of the invention the microorganism is Escherichia such as E. coli K12, Bacillus, Pseudomonas, Rhodococcus, or Actinomyces. In one embodiment of the invention the microorganism is non-pathogenic.

In one embodiment of the invention the conversion of a starting substrate, such as D-glucose, to xylitol, xylose, or xylulose occurs by a single recombinant or isolated microorganism. In another embodiment of the invention two or more recombinant microorganisms can be used in the conversion of the substrate to xylitol. Each of the microorganisms can be capable of completely converting the substrate to xylitol, xylose, or xylulose or a combination thereof. Alternatively, one or more microorganisms can perform one or more steps of this pathway, while one or more other microorganisms can perform one or more steps of the pathway wherein an end-product of xylitol, xylose, or xylulose or a combination thereof is produced. Optionally, a mixture of microorganisms that can perform one or more steps of the pathway are used.

In one embodiment of the invention an end-product of D-xylose is produced by a microorganism that has transketolase, xylulokinase (or other xylulose 5-phosphate dephosphorylating activity), and xylose isomerase activities, but that lacks or has reduced xylose reductase and xylose dehydrogenase activities as compared to a wild-type microorganism. The microorganism can have elevated transketolase, xylulokinase ((or other xylulose 5-phosphate dephosphorylating activity), and xylose isomerase activities as compared to a wild-type microorganism.

In another embodiment of the invention an end-product of D-xylulose is produced by a microorganism that has transketolase and xylukinase activities (or other xylulose 5-phosphate dephosphorylating activity), but that lacks or has reduced xylose reductase, xylose dehydrogenase, and xylose isomerase activities as compared to a wild-type microorganism. The microorganism can have elevated transketolase and xylukinase activities (or other xylulose 5-phosphate dephosphorylating activities) as compared to a wild-type microorganism.

E. coli tktA, xylB and xylA genes are a suitable source for TK, XK and XI, respectively, since all have previously been cloned and characterized and sequence data is available. S. cerevisiae may be used as an alternative source for XK as the XKS1 gene product has been shown to have greater xylulose-5-phosphate dephosphorylation activity that the E. coli XK. Although xylitol dehydrogenase (XDH) has a catabolic role in vivo the enzyme also functions as a reductase and could be used in the final step of the pathway. Many other microbial genera can be used as sources for the enzymes due to the specific activity of the individual enzymes. These include but are not limited to: Escherichia, Bacillus, Pseudomonas, Rhodococcus, Actinomyces, yeast. XR may be obtained from E. coli or P. stipitis as described in co-pending U.S. patent application Ser. No. ______, filed May 19, 2005, entitled “Methods for Production of Xylitol in Microorganisms” (incorporated herein by reference in its entirety), or from other microbial sources. Xylose reductases generally have broad substrate specificities and function on both D-xylose as well as L-arabinose (Hahn-Hagerdal, Jeppsson et al. 1994; Richard, Verho et al. 2003). Many sources of xylose reductases are suitable for use. In one embodiment of the invention, a xylose reductase of Pichia stipitis is used because its DNA sequence is available, it can use both NADH and NADPH as enzyme cofactor and has good activity on both L-arabinose and D-xylose. Two putative xylose reductases from E. coli (yafB and yajO) could also be used due to the ease with which they can be cloned and expressed in E. coli. XYL1 from Candida tenuis can also be used.

XDH can be obtained from many microbial sources including Gluconobacter sp. and Tricoderma reesei as described in co-pending U.S. patent application Ser. No. ______, filed May 19, 2005, entitled “Methods for Production of Xylitol in Microorganisms”.

In one embodiment of the invention, the nucleic acid sequence encoding xylose isomerase and xylulokinase can be an Escherichia coli xylAB operon. Xylulokinase activity can also be proved by another D-xylulose 5-phosphate dephosphorylating enzyme nucleic acid sequence such as the nucleic acid sequence of Mycobacterium sp. or Pichia angusta dihydroxyacetone synthase or Escherichia coli alkaline phosphatase.

In one embodiment of the invention the pathway described above can be constructed in a single microorganism, such as a bacterial strain, with the expression of the enzyme genes being driven by exogenous constitutive or inducible promoters either on multi-copy plasmids or in the chromosome of the host strain. E. coli K-12 can be used as a host due to ease of manipulation but other microorganisms can also be used.

In one embodiment of the invention a recombinant microorganism expresses transketolase (TK), xylulokinase (XK), xylose isomerase (XI) and aldose (xylose) reductase (XR) and optionally xylitol dehydrogenase (XDH) activities. In another embodiment of the invention, a recombinant microorganism comprises transketolase, xylulokinase, and xylitol dehydrogenase activities. These activities can be naturally present in the microorganism (i.e., wild-type) or can be recombinant activities (i.e., a heterologous nucleic acid sequence is added to the microorganism and is expressed by the microorganism). The recombinant microorganism can comprise one or more recombinant nucleic acid sequences encoding, for example, transketolase (TK), xylulokinase (XK), xylose isomerase (XI) and aldose (xylose) reductase (XR) and optionally xylitol dehydrogenase (XDH). Methods of making recombinant microorganisms are well known in the art. See e.g., Sambrook et al., Molecular Cloning: A Laboratory Manual, Cold Spring Harbor Laboratory, New York (1989), Current Protocols in Molecular Biology, Ausebel et al (eds), John Wiley and Sons, Inc. New York (2000). Furthermore, methods of constructing recombinant microorganisms are described in the Examples below.

One embodiment of the invention provides a method for producing a xylitol end-product comprising fermenting a substrate comprising D-glucose with a recombinant microorganism of the invention. D-fructose-6-phosphate, D-xylulose-5-phosphate, D-xylulose, D-xylose, or combinations thereof can be produced as intermediates to the xylitol end-product.

In one embodiment of the invention, hexoses other than D-glucose or in combination with D-glucose such as maltose, lactose, D-fructose, D-mannose, L-sorbose, D-glucosamine, melibose, and galactose can be used as a substrate. Combinations of these substrates can also be used.

Screening Methods and Strains

Xylose Reductase Screening Strains.

An important part of this invention is the development of suitable screening strains for detection and enhancement of the individual enzymes. A screening strain can comprise a bacterium such as E. coli K12 strain carrying a xylose isomerase deletion (xylAΔ) thus making it unable to grow on and utilize D-xylose as a carbon source. E. coli cannot synthesize or utilize xylitol as a carbon source, and addition of a deregulated xylitol dehydrogenase gene into this host strain would enable growth on xylitol because the XDH will convert xylitol to D-xylulose, which can then be utilized via intermediary metabolism. It follows that this strain when transformed with a plasmid carrying a putative xylose reductase gene could be used to screen for XR reductase activity. That is, active clones when grown on a D-xylose minimal medium will only grow if the D-xylose is converted to xylitol. Such a strain would be very useful for cloning novel aldose reductases, preliminary screening of mutagenesis libraries and could also be adapted into a high throughput plate screen for evolved reductases. A number of suitable XR screening strains have been described in co-pending U.S. application Ser. No. ______, filed May 19, 2005, entitled “Methods for Production of Xylitol in Microorganisms”.

High Throughput in Situ Screens for, i) Strains Producing Xylitol and/or Xylose from D-Glucose and ii) the Detection of Genes Encoding Xylose Reductase.

The rational design of a novel biosynthetic pathway to xylitol from D-glucose can be significantly enhanced by the construction of a high throughput screen to detect rate limiting steps in the pathway and random mutational events which lead to increased production of xylitol. In this way the novel xylitol pathway can benefit from strain improvement regimes which have proven highly successful for other compounds produced by microbial fermentation, such as amino acids and secondary metabolites. The basis of the high throughput screen for strains producing xylitol from D-glucose is a solid phase crossfeeding assay in which the growth of an indicator strain, embedded in a solid medium such as an agar plate, is dependent upon the synthesis and exodus of xylitol from individual colonies of the xylitol producing strain, plated onto the agar, such that zones of “crossfed” indicator strain growth will surround plated colonies which produce xylitol. The screen is calibrated to be sufficiently quantitative to identify those crossfeeding colonies which generate the largest zones of surrounding growth and are therefore producing the highest titres of xylitol.

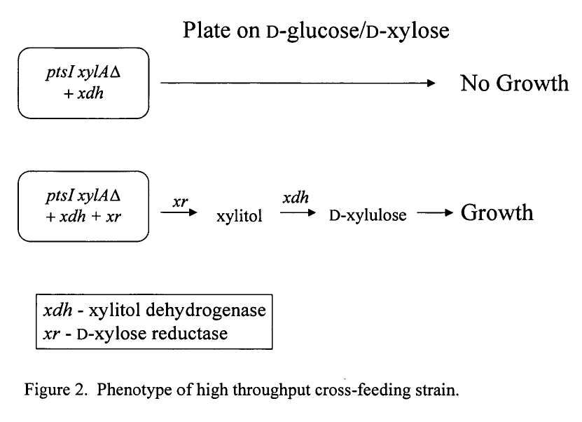

The indicator strain can be an E. coli ptsI, xylA deletion mutant which carries a constitutively expressed xylitol dehydrogenase (XDH) from a heterologous source such as Gluconobacter oxydans or T. reesei (see, e.g., co-pending U.S. application Ser. No. ______, filed May 19, 2005, entitled “Methods for Production of Xylitol in Microorganisms”). Such a mutant cannot utilize glucose or xylose as a primary carbon source but can metabolize xylitol via XDH (FIG. 2) and the pentose phosphate pathway. As an intermediate step, this screen can be adapted such that an E. coli strain carrying only the ptsI deletion mutation and similarly embedded in a solid medium such as an agar can be used to detect colonies plated onto the agar which produce xylose or xylitol or a combination thereof from D-glucose. The screen can also be applied to identify and isolate genes encoding xylitol reductase as the incorporation of this activity into the ptsI, xylA background enables growth of the strain on xylose as sole carbon source.

In one embodiment of the invention, a recombinant indicator microorganism expresses substantially no phosphotransferase enzyme I and substantially no xylose isomerase and has xylitol dehydrogenase activity. The microorganism can be a phosphotransferase enzyme I deletion mutant or a xylose isomerase deletion mutant or both. The indicator microorganism can comprise a recombinant nucleic acid sequence encoding xylitol dehydrogenase such as a Gluconobacter or Tricoderma reesi nucleic acid sequence. The indicator microorganism can be a bacterium, such as E. coli, yeast or fungi. The indicator microorganism can be Lac⁺.

To set up a screen an indicator strain is seeded at an appropriate density into agar plates containing M9 minimal glucose medium and the candidate xylitol-producing clones are spread onto the plates, where they will convert the D-glucose to xylitol. Indicator strain cells in the vicinity of the xylitol producing clones will cross-feed on the xylitol produced and generate a growth zone. To improve the clarity of the growth zones the indicator strain can be made lac⁺ while the xylitol-producing strains are lac⁻ (any reporter gene could be used). In this case the cross-fed zones will appear dark blue on plates supplemented with the lactose analogue X-gal. Those clones that produce the highest titer of xylitol will give rise to the largest cross-feeding colonies, allowing easy visualization of the relative levels of xylitol production by individual mutants. This screen will enable a throughput of approximately 10,000 colonies per plate on 8-inch Petri dishes, facilitating screening of approximately 10⁶ colonies in a single experiment.

In order to increase visualization of the indicator microorganisms, the beta-galactosidase enzyme of the indicator microorganism can be more tightly regulated that a wild-type beta-galactosidase enzyme by, for example, elevating the intracellular level of a lactose repressor protein in the indicator microorganism. Alternatively, the beta-galactosidase enzyme of the indicator microorganism can possess a shorter half-life than a wild-type beta-galactosidase due to alterations to its peptide sequence that decreases its stability under physiological conditions. See, e.g., DNA sequence for a low-level promoter of the lac repressor gene and an ‘up’ promoter mutation. Calos M P, Nature 1978 274(5673): 762-5; Multicopy expression vectors carrying the lac repressor gene for regulated high-level expression of genes in Escherichia coli. Stark M J R, Gene 1987 51(2-3): 255-267; Role of a peptide tagging system in degradation of proteins synthesized from damaged messenger RNA. Science. 1996 Feb. 16;271(5251):990-3; Sequence determinants of C-terminal substrate recognition by the Tsp protease. J Biol Chem. 1996 Feb. 2;271(5):2589-93; New unstable variants of green fluorescent protein for studies of transient gene expression in bacteria. Appl Environ Microbiol. 1998 June;64(6):2240-6.

The xylitol-producing strains can be subjected to random mutagenesis using agents including but not limited to NTG or nitrous acid or the plasmid DNA or individual cloned genes can be subjected to random mutagenesis using mutator strains such as XL1-Red (Stratagene, La Jolla, Calif.) or error prone PCR and re-introduced to the production host. Strains that demonstrate superior performance in xylitol production can be subjected to iterative improvement using repeated rounds of mutagenesis and screening. Additionally, isolates displaying increased xylitol production in each round of the cross-feeding screen can be analyzed by high performance anion exchange chromatography or high performance liquid chromatography, or assayed, for the xylitol intermediates D-xylose, D-xylulose and D-xylulose-5-phosphate. This will enable changes in the flux of carbon to xylitol to be correlated with the activity of particular enzymes. By comparing the activities of the corresponding enzymes in parent strains, the effect of random mutations upon rate limiting steps in the pathway can be assessed. Where the activities of particular enzymes are significantly affected, the respective genes can be sequenced and the nature of any mutation(s) characterized. Where increased xylitol production results from alteration in the expression of a particular enzyme the expression can be modulated using a rational approach to investigate the effect. The cross-feeding screen will also enable the identification of mutants that display faster growth rates while maintaining a high level of xylitol production. In this way the integration of the rational and random mutational approaches to the development of xylitol producing strains can be used to maximum benefit.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

The invention illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

### EXAMPLES

### Example 1

### Construction of a Xylose Biosynthetic Pathway Comprising the E. coli Transketolase, Xylulokinase and Xylose Isomerase Genes Cloned onto a Single Expression Vector for Expression in E. coli

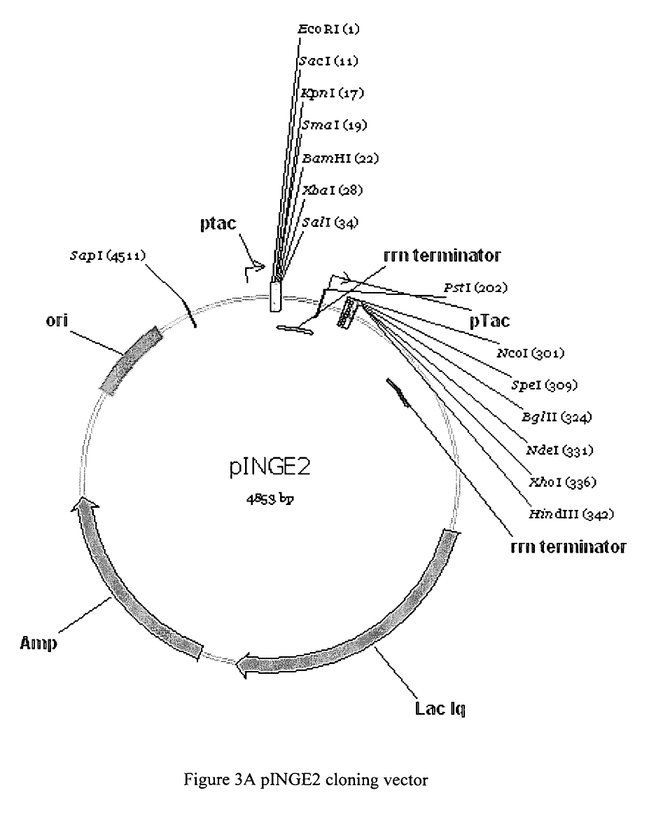

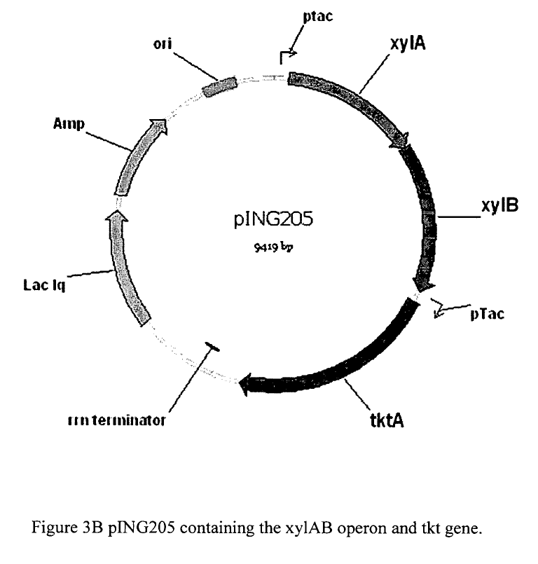

An Escherichia coli tktA gene encoding TK and the xylAB operon encoding XI and XK were cloned from the genomic of DNA of E. coli K12 (XL1-blue, Stratagene) using primers designed from the published sequences (Genbank accession numbers X68025, K01996 and Table 1). E. coli was grown overnight in 2 ml LB medium and the genomic DNA isolated using the Sigma GenElute kit (Sigma, UK). The genes were amplified using Pfu polymerase (Sigma, UK) and standard reaction components in an Eppendorf Mastercycler PCR machine. The reaction products were isolated then restricted with NcoI and XhoI (tktA) or EcoRI and XbaI (xylAB) using standard conditions before being sequentially ligated into the correspondingly cleaved expression vector pINGE2 restricted with the same enzymes to give the plasmid pING205 (FIG. 3) This vector is then used to transform appropriate host strains of E. coli for the production of xylitol from D-glucose. The resulting recombinant strains can be cultured in shake flasks e.g. 100 ml LB culture, plus appropriate antibiotics for plasmid maintenance, in a 1 L shake flask. Samples taken throughout the growth and pre- or post-induction of enzyme biosynthesis can then be subjected to analysis by HPLC for the presence of xylose or intermediates in the novel biosynthetic pathway.

### Example 2

### Construction of a Xylitol Biosynthetic Pathway Comprising the E. coli Xylulokinase and Xylose Isomerase Genes and C. tenuis Xylose Reductase Gene Cloned onto a Single Expression Vector and Expressed in E. coli Along with the E. coli Transketolase Gene Cloned onto a Compatible Expression Vector and Co-Expressed in the Same Organism

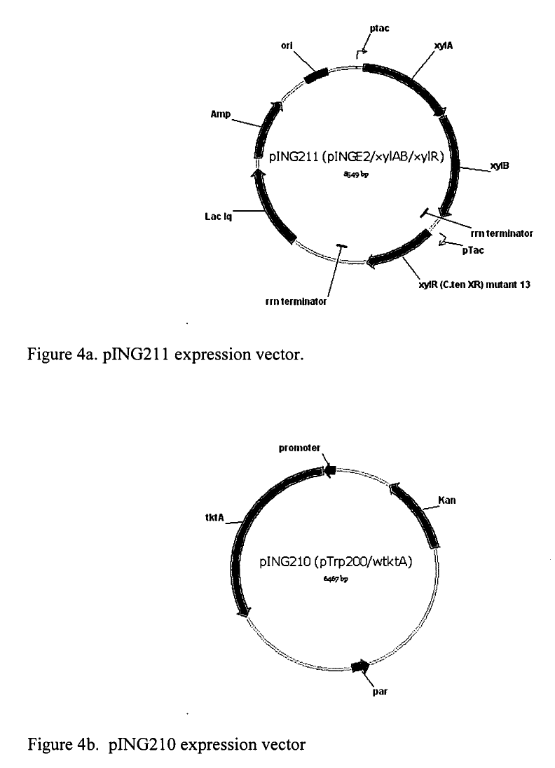

An Escherichia coli xylAB operon encoding XI and XK was isolated from the genomic of DNA of E. coli K12 and cloned into the first multiple cloning site of pINGE2 as described in Example 1. The xylR of C. tenuis encoding xylose reductase was amplified by PCR and flanked by sites for the restriction enzymes NcoI and HindIII. This was then ligated into the second multiple cloning site of pINGE2 to give plasmid pING211 as shown in FIG. 4a. This vector was then used to transform appropriate host strains of E. coli. A second compatible plasmid, pING210 (FIG. 4b), based on the low-copy number vector pTrp200 and containing the E. coli tktA gene encoding transketolase expressed from its native promoter was then used to transform strains carrying pING211 to give a two plasmid synthetic pathway to xylitol from D-glucose. The resulting recombinant strains can be cultured in shake flasks e.g. 100 ml LB culture, plus appropriate antibiotics for plasmid maintenance, in a 1 L shake flask. Samples taken throughout the growth and pre- or post-induction of enzyme biosynthesis can then be subjected to analysis by HPLC for the presence of xylitol or intermediates in the novel biosynthetic pathway.

### Example 3

### High Throughput Screen for Xylose or Xylitol Producing Strains

The ptsI E. coli strain PP2418 that is unable to grow on D-glucose was obtained from the Coli Genetic Stock Centre. The strain was transformed with plasmid pZUC15 containing xylitol dehydrogenase from Gluconobacter oxydans as in example 10 to produce the screening strain OR13. The ability of the strain to metabolize and grow upon xylitol while showing negligible growth on glucose was confirmed in growth assays in which the strain was plated on M9 minimal agar plates containing 0.2% D-glucose or 0.2% xylitol as sole carbon source This strain can be used in a crossfeeding assay to detect strains that overproduce xylitol or D-xylose using D-glucose as sole carbon source. The strain is seeded at a density of 2.5×10⁸ cells/ml in cooled M9 minimal agar cooled to 45° C. and containing 0.2% glucose, 1 mM MgSO₄ and 0.1 mM CaCl₂. The seeded mix is poured into Petri dishes to form a solid medium for colony plating. Strains to be assayed visually for xylose or xylitol production are then plated onto the agar and the production of xylose or xylitol visualized by surrounding zones of indicator strain growth zones due to crossfeeding by xylose or xylitol. The extent of xylose or xylitol biosynthesis can be quantitated by the size of the resulting crossfeeding zone.

By deletion of the xylA gene from this strain, a high throughput screen linked to the conversion of D-xylose to xylitol could also be developed. PP2418 xylAΔ carrying pZUC15 (xdh) will not grow on D-xylose or glucose but will grow on xylitol. Thus mutated aldose reductases could be selected using a crossfeeding screen on plates containing D-xylose, i.e. the more xylitol produced by the mutated aldose reductase the larger the crossfeeding zone produced by PP2418 xylAΔ carrying pZUC15. Similarly strains producing xylitol from dextrose can be directly assayed for xylitol production using this indicator strain in the assay described in Example 3. Indicator strain growth will only be supported by xylitol and not xylose due to the lack of efficient xylose reductase in E. coli K12 and the deletion of the xylose isomerase gene. The crossfeeding assay is carried out using indicator strain seeding density and media formulation as described in Example 3.

TABLE 1
List of DNA PCR primers.

| | | | |
|---|---|---|---|
| Enzyme | Organism | Forward Primer | Reverse Primer |

| | | | |
|---|---|---|---|
| XylAB | E. coli K12 | SEQ ID NO:1 | SEQ ID NO:2 |
| | | AGCATGAATTCGCAAG | GAGAGTCTAGATTACG |
| | | CCTATTTTGACCAGCT | CCATTATTGCCAGAAG |
| | | CGAT | TTGC |
| TktA | E. coli K12 | SEQ ID NO:3 | SEQ ID NO:4 |
| | | GCGCCATGGCTTCCTC | GAGAGTCTAGATTACA |
| | | ACGTAAAGAGCTTGCC | GCAGTTCTTTTGCTTT |
| | | | CGC |

We claim:

1. A recombinant microorganism comprising a recombinant biochemical pathway to produce xylose or xylitol from fermentation of D-glucose.

2. A recombinant microorganism comprising transketolase, xylulokinase, xylose isomerase, and xylose reductase activities, wherein one or more of the transketolase, xylulokinase, xylose isomerase, and xylose reductase activities are elevated as compared to a wild-type microorganism, and wherein the recombinant microorganism can produce an end-product of xylose, xylitol, or a combination thereof from a substrate comprising D-glucose.

3. The recombinant microorganism of claim 2, wherein the microorganism further comprises xylitol dehydrogenase activity.

4. The recombinant microorganism of claim 3, wherein the xylitol dehydrogenase activity is elevated as compared to a wild-type microorganism.

5. The recombinant microorganism of claim 2, wherein the xylose isomerase activity is reduced or eliminated as compared to a wild-type microorganism.

6. The recombinant microorganism of claim 2, wherein the microorganism is a bacterium, yeast or fungus.

7. The recombinant microorganism of claim 2, wherein the microorganism is Escherichia, Bacillus, Pseudomonas, Rhodococcus, or Actinomyces.

8. The recombinant microorganism of claim 2, wherein the recombinant microorganism comprises one or more recombinant nucleic acid sequences encoding transketolase, xylulokinase, xylose isomerase, and xylose reductase.

9. The recombinant microorganism of claim 2, wherein the xylulokinase activity is replaced by a D-xylulose 5′ phosphate dephosphorylating enzyme.

10. The recombinant microorganism of claim 3, wherein the recombinant microorganism comprises one or more recombinant nucleic acid sequences encoding transketolase, xylulokinase, xylose isomerase, xylose reductase, a D-xylulose 5′ phosphate dephosphorylating enzyme, and xylitol dehydrogenase.

11. The recombinant microorganism of claim 10, wherein the recombinant nucleic acid sequence encoding xylose reductase is a Pichia stipitis nucleic acid sequence.

12. The recombinant microorganism of claim 10, wherein the nucleic acid sequence encoding xylose reductase comprises a nucleic acid sequence encoding XYL1 from Candida tenuis.

13. The recombinant microorganism of claim 10, wherein the nucleic acid sequence encoding xylose reductase comprises a yafB or yajO nucleic acid sequence from E. coli.

14. The recombinant organism of claim 10, wherein the nucleic acid sequence encoding xylitol dehydrogenase is a Gluconobacter or Tricoderma reesi nucleic acid sequence.

15. The recombinant organism of claim 10, wherein the nucleic acid sequence encoding the D-xylulose 5′ phosphate dephosphorylating enzyme is an E. coli alkaline phosphatase nucleic acid sequence.

16. The recombinant organism of claim 10, wherein the nucleic acid sequence encoding the D-xylulose 5′ phosphate dephosphorylating enzyme is a Mycobacterium sp. or Pichia angusta dihydroxyacetone synthase nucleic acid sequence.

17. The recombinant organism of claim 10, wherein the nucleic acid sequence encoding transketolase is an Escherichia coli tktA nucleic acid sequence.

18. The recombinant organism of claim 10, wherein the nucleic acid sequence encoding xylose isomerase is an Escherichia coli xylA nucleic acid sequence.

19. The recombinant organism of claim 10, wherein the nucleic acid sequence encoding xylulokinase is an Escherichia coli xylB nucleic acid sequence or a Saccharomyces cerevisiae XKS 1 nucleic acid sequence.

20. The recombinant organism of claim 10, wherein the nucleic acid sequence encoding xylose isomerase and xylulokinase is an Escherichia coli xylAB operon.

21. The recombinant microorganism of claim 2, wherein the recombinant microorganism is non-pathogenic.

22. The recombinant microorganism of claim 2, wherein the recombinant microorganism produces D-fructose-6-phosphate as an intermediate to the xylitol or xylose end-product.

23. The recombinant microorganism of claim 2, wherein the recombinant microorganism produces D-xylulose-5-phosphate as an intermediate to the xylitol or xylose end-product.

24. The recombinant microorganism of claim 2, wherein the recombinant microorganism produces D-xylulose as an intermediate to the xylitol or xylose end-product.

25. The recombinant microorganism of claim 2, wherein the recombinant microorganism produces D-xylose as an intermediate to the xylitol or xylose end-product.

26. A method for producing xylitol, xylose or a combination thereof end-product comprising fermenting a substrate comprising D-glucose with the recombinant microorganism of claim 2.

27. The method of claim 26, wherein D-fructose-6-phosphate is produced as an intermediate to the xylitol, xylose or combination thereof end-product.

28. The method of claim 26, wherein D-xylulose-5-phosphate in produced as an intermediate to the xylitol, xylose or combination thereof end-product.

29. The method of claim 26, wherein D-xylulose in produced as an intermediate to the xylitol, xylose or combination thereof end-product.

30. The method of claim 26, wherein D-xylose in produced as an intermediate to the xylitol end-product.

31. The method of claim 26, wherein D-fructose-6-phosphate, D-xylulose-5-phosphate, D-xylulose, and D-xylose are produced as intermediates to the xylitol end-product.

32. A method for producing xylitol, xylose or a combination thereof end-product comprising fermenting D-glucose with the recombinant microorganism of claim 2.

33. A recombinant indicator microorganism that expresses substantially no phosphotransferase enzyme I, expresses substantially no xylose isomerase, and has xylitol dehydrogenase activity.

34. The recombinant indicator microorganism of claim 33, wherein the microorganism is lac+.

35. The recombinant indicator microorganism of claim 33, wherein the recombinant indicator microorganism comprises a recombinant nucleic acid sequence encoding xylitol dehydrogenase.

36. The recombinant indicator microorganism of claim 33, wherein the nucleic acid sequence encoding xylitol dehydrogenase is a Gluconobacter or Tricoderma reesi nucleic acid sequence.

37. The recombinant indicator microorganism of claim 33 wherein the microorganism is a bacteria, yeast or fungi.

38. The recombinant indicator microorganism of claim 33, wherein the microorganism is E. coli.

39. The recombinant indicator microorganism of claim 33, wherein the microorganism is a phosphotransferase enzyme I deletion mutant or a xylose isomerase deletion mutant or both a phosphotransferase enzyme I deletion mutant and a xylose isomerase deletion mutant.

40. A method of detecting production of xylose, xylulose or xylitol from a sole carbon source by a microorganism, comprising i) embedding the recombinant indicator microorganism of claim 33 in a solid medium comprising D-glucose as a sole carbon source, ii) plating microorganisms to be tested on the solid media and incubating the solid media under conditions suitable for growth of the indicator microorganism and the microorganisms to be tested, wherein one or more plated colonies producing xylose or xylitol are visualized by growth of the indicator strain in an area surrounding the colony.

41. A method of detecting the production of xylose from a sole carbon source by a microorganism, comprising i) embedding the recombinant indicator microorganism of claim 33 in a solid medium comprising D-glucose as a sole carbon source, ii) plating microorganisms to be tested on the solid media and incubating the solid media under conditions suitable for growth of the indicator microorganism and the microorganisms to be tested, wherein one or more plated colonies producing xylose are visualized by growth of the indicator strain in an area surrounding the colony.

42. A method of detecting production of xylitol from a sole carbon source by a microorganism, comprising i) embedding the recombinant indicator microorganism of claim 33 in a solid medium comprising D-glucose as a sole carbon source, ii) plating microorganisms to be tested on the solid media and incubating the solid media under conditions suitable for growth of the indicator microorganism and the microorganisms to be tested, wherein one or more plated colonies producing xylitol are visualized by growth of the indicator strain in an area surrounding the colony.

43. The method of claim 40 wherein the microorganism to be tested is subjected to random mutation using biological, chemical or physical means prior to the plating.

44. The method of claim 40 wherein the indicator microorganism is lac⁺ and the microorganism to be tested for production of xylitol is lac⁻ and wherein the solid media comprises X-gal, wherein areas of growth of the indicator microorganism are blue.

45. The method of claim 40 wherein the beta-galactosidase enzyme of the indicator microorganism is more tightly regulated than a wild-type beta-galactosidase enzyme by elevation of the intracellular level of a lactose repressor protein.

46. The method of claim 40 wherein the beta-galactosidase enzyme of the indicator microorganism possesses a shorter half-life than wild type beta-galactosidase due to alterations to its peptide sequence that decrease its stability under physiological conditions.

47. A recombinant E. coli strain that produces substantially no phosphotransferase enzyme I and produces substantially no xylose isomerase.

48. The recombinant E. coli strain of claim 47, wherein the E. coli strain is a phosphotransferase enzyme I deletion mutant or a xylose isomerase deletion mutant or both a phosphotransferase enzyme I deletion mutant and a xylose isomerase deletion mutant.

49. A method for screening for xylitol reductase activity comprising: transforming the E. coli strain of claim 47 with a nucleic acid molecule encoding a putative xylose reductase to produce a transformant, and adding the transformant to a media comprising xylose as the sole carbon source, wherein if the transformant comprises an expressed nucleic acid encoding a xylose reductase, then the transformant will grow in the media.

50. A method for production of xylitol, xylose, or combinations thereof from simple sugars via d-fructose-6-phosphate and d-xylulose-5-phosphate intermediates comprising:
(a) converting the simple sugars to D-fructose-6-phosphate with glycolysis performed with a microorganism;
(b) converting the D-fructose-6-phosphate to D-xylulose-5-phosphate with transketolase;
(c) converting D-xylulose-5-phosphate to D-xylulose with xylulokinase
(d) converting the D-xylulose to:
(i) xylitol with xylitol dehydrogenase; or
(ii) D-xylose with xylose isomerase; and converting the D-xylose to xylitol with xylose reductase; or
(iii) a combination of (i) and (ii).


SEQUENCE LISTING (4 sequences)

SEQ ID NO: 1 - DNA, 36 bp, Escherichia coli
agcatgaattcgcaagcctattttgaccagctcgat

SEQ ID NO: 2 - DNA, 36 bp, Escherichia coli
gagagtctagattacgccattattgccagaagttgc

SEQ ID NO: 3 - DNA, 32 bp, Escherichia coli
gcgccatggcttcctcacgtaaagagcttgcc

SEQ ID NO: 4 - DNA, 35 bp, Escherichia coli
gagagtctagattacagcagttcttttgctttcgc